(19) United States
(12) Patent Application Publication
(10) Pub. No.: US 2008/0257978 A1
(43) Pub. Date: Oct. 23, 2008

(54) Fragrance Dispenser

(76) Inventors: Conroy R. Marth, Preston, MD (US); Ernest Edmond Dill, Viola, DE (US); James Edward Thompson, Easton, MD (US)

Correspondence Address: KILPATRICK STOCKTON LLP, 1001 WEST FOURTH STREET, WINSTON-SALEM, NC, 27101, US

(21) Appl. No.: 12/023,779

(22) Filed: Jan. 31, 2008

Related U.S. Application Data
(60) Provisional application No. 60/898,883, filed on Jan. 31, 2007.

(51) Int. Cl. A61L 9/04; B23P 17/00

(52) U.S. Cl. 239/60; 29/592

(57) ABSTRACT

Fragrance dispenser products and methods of manufacture are disclosed. For example, one embodiment of a fragrance dispenser according to the present invention includes a container having an inner surface and an outer surface, the inner surface defining a cavity; a quantity of gel disposed within the cavity, the quantity of gel having a fragrance and configured to evaporate when exposed to air, wherein the container is configured to engage with a mounting bracket; and at least one opening defined by and extending through the outer and inner surfaces, the opening configured to expose the quantity of gel to air and to provide a visual indication of the quantity of gel.

### CROSS-REFERENCE TO RELATED APPLICATIONS

The present application hereby claims priority to U.S. Provisional Patent Application Ser. No. 60/898,883, entitled “Fragrance Dispenser,” filed Jan. 31, 2007, which is incorporated herein by reference in its entirety.

### FIELD OF THE INVENTION

The present invention generally relates to fragrance dispensers.

### BACKGROUND

Fragrances have been used to combat odors in a variety of settings. For example, candles have been used to provide a more pleasing scent than otherwise might be present in an environment, such as in a bathroom or lavatory. And air fresheners have been used in airline lavatories to reduce odors in the lavatory and the rest of the airplane. Some conventional fragrance dispensers are made of a porous material formed in a puck shape with a protrusion on one side so as to engage with a mounting bracket. The puck is saturated with fragrance and installed within a mounting bracket. The fragrance slowly dissipates into the environment to provide odor control.

Such standard porous pucks are disadvantageous for several reasons. For instance, the porous material must be directly contacted by personnel, thus exposing the personnel to the chemicals saturated within the puck, or mandating the use of gloves to handle the material. Also, there is no indication as to the amount of fragrance remaining, and attempts to use a fragrance material that fades in color have generally proved unsuccessful.

### SUMMARY

Embodiments of the present invention provide methods and apparatuses for dispensing fragrance. For example, in one embodiment, a device for delivering fragrance comprises a container having an inner surface and an outer surface where the inner surface defines a cavity. A quantity of gel is disposed within the cavity, the quantity of gel having a fragrance and configured to evaporate when exposed to air. Further, the container of such an embodiment configured to engage with a mounting bracket and have at least one opening defined by and extending through the outer and inner surfaces. The opening is configured to expose the quantity of gel to air and to provide a visual indication of the remaining quantity of gel.

This illustrative embodiment is mentioned not to limit or define the invention, but to provide an example to aid understanding thereof. Illustrative embodiments are discussed in the Detailed Description, and further description of the invention is provided there. Advantages offered by various embodiments of this invention may be further understood by examining this specification.

### BRIEF DESCRIPTION OF THE DRAWINGS

These and other features, aspects, and advantages of the present invention are better understood when the following Detailed Description is read with reference to the accompanying drawings, wherein:

FIG. 1 is diagrams of a fragrance dispenser according to one embodiment of the present invention;

FIG. 2 shows a mounting bracket suitable for use with a fragrance dispenser according to one embodiment of the present invention.

FIG. 3 shows a fragrance dispenser 100 according to one embodiment of the present invention engaged with a mounting bracket 200; and

FIG. 4 shows a flow chart illustrating a method for manufacturing a fragrance dispenser according to one embodiment of the present invention.

### DETAILED DESCRIPTION

Embodiments of the present invention provide devices and methods of manufacture for providing a fragrance dispenser.

### Illustrative Fragrance Dispenser

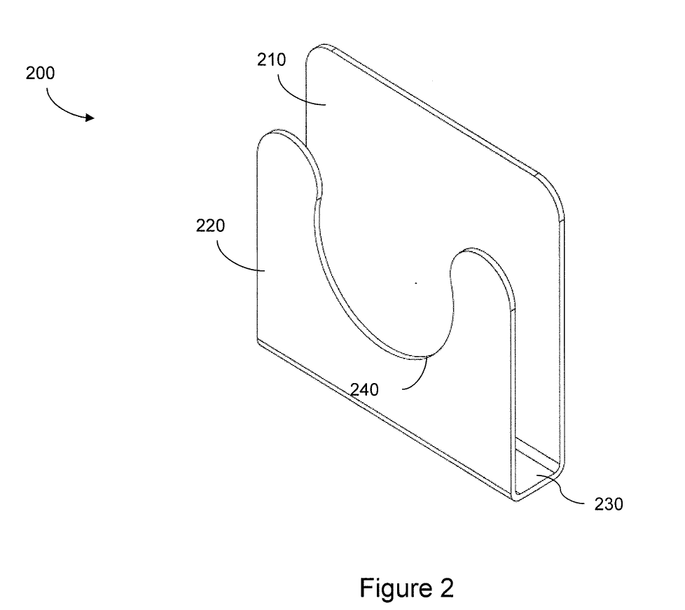

In one embodiment of the present invention, a lavatory in an airplane has been fitted with a mounting bracket for accepting and holding a fragrance dispenser. For example, a typical airline industry mounting bracket may have a shape substantially as shown in FIG. 2. The bracket has three faces: a rear face configured to be mounted on a wall or other vertical surface; a front face substantially parallel with the rear face and having a substantially U-shaped cut out; and a bottom face substantially perpendicular and affixed to the rear and front faces.

The illustrative fragrance dispenser is made of a plastic material and has a substantially disc shape having front and rear faces. The front face has a circular projection approximately in the center of the front face that is configured to engage with the U-shaped cut-out in the mounting bracket. The circular projection comprises a wall surrounding a circular hole formed in the front face of the fragrance dispenser, such that the wall engages with the U-shaped cut-out and allows the circular hole to be exposed to the environment in the lavatory. By engaging the U-shaped cut-out, the fragrance dispenser may be prevented from rolling out from between the faces of the mounting bracket if the plane enters a turn, pitches backwards or forwards during takeoff or landing, or otherwise moves in a manner that might cause the fragrance dispenser to slide or roll our of the mounting bracket. Thus, the illustrative fragrance dispenser may be well-suited for use in existing airline standard mounting bracket. Further, the illustrative fragrance dispenser provides better performance, such as more effective and efficient dispersal of fragrance into an environment, than existing fragrance dispensers due to its shape, the location of openings to expose the fragrance to the environment, and the reduced chance of user contact with the fragrance material itself.

### Fragrance Dispenser

Embodiments of the invention presented herein provide an enhanced fragrance dispenser.

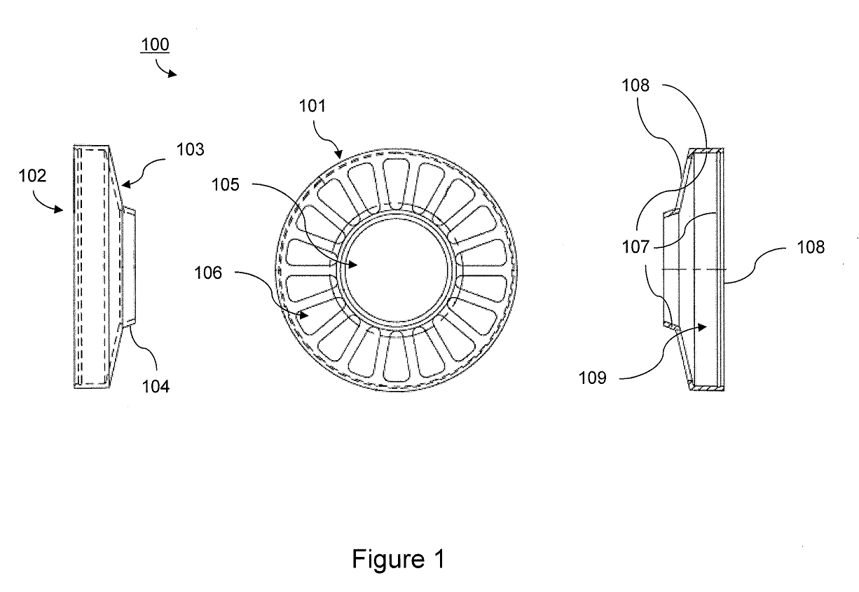

Referring now to the drawings in which like numerals indicate like elements throughout the several Figures, FIG. 1 is a diagram of a fragrance dispenser 100 according to one embodiment of the present invention. Fragrance dispenser 100 comprises a container 100 having an inner surface 107 and an outer surface 108. The inner surface 107 of the container defines a cavity 109. Disposed within the cavity 109 is a quantity of gel having a fragrance, wherein the gel is configured to evaporate when exposed to air. In the embodiment shown in FIG. 1, the container has at least one opening 105, 106 defined by and extending through the inner and outer surfaces 107, 108. Opening 105 has a substantially circular shape and is configured to expose the cavity 109 to the environment outside of the container 100 and to allow air to pass into and out of the cavity 109 and to expose the gel to the air.

One embodiment of the present invention may further comprise a foot or other protrusion within the container that may be obscured by the gel, and may provide an indication of an amount of the gel remaining, based at least upon the amount of the foot exposed. For example, an inner surface of the back face 102 may comprise a protrusion that extends into the cavity 109, referred to as a foot. The foot may be configured to be covered by gel when a quantity of gel is inserted into the cavity 109. As the gel is depleted during the use, the foot may become exposed and may provide an indication that the air freshener may be replaced. In one embodiment, the protrusion may comprise a color different than the gel, which may aid in seeing the foot as the gel is depleted. In one embodiment, the foot may not comprise any additional coloring beyond the base color of the material from which the foot is formed.

The container 100 shown in FIG. 1 comprises a front face 103 coupled to a back face 102 to form cavity 109. In the embodiment shown, front face 103 and back face 102 each comprise a substantially circular shape. Front face 103 comprises a first diameter and back face 102 comprises a second diameter. In the embodiment shown, front face 103 and back face 102 have substantially the same diameter. However, in other embodiments, front face 103 and back face 102 may have a different diameter. In one embodiment front face 103 and the back face 102 are formed as a unitary structure. In another embodiment, the back face 102 is configured to be coupled to the front face, such as by an interference fit, with an adhesive, or with a locking mechanism such as locking tabs.

According to other embodiments, a fragrance dispenser may comprise a single piece of material molded to form an inner surface and an outer surface. For example, in one embodiment of the present invention, a fragrance dispenser may be injection molded in a single piece. Still further embodiments of the present invention may comprise a plurality of components that may be fitted together to create a fragrance dispenser. For example, according to one embodiment of the present invention, a fragrance dispenser may comprise a back face, a ring, and a front face. The back face may couple to the ring, which may have a diameter substantially the same as the diameter of the back face. The front face may couple to the ring as well, the front face having a diameter substantially the same as the ring's diameter. By coupling the three components, an inner surface and an outer surface may be formed. Still further embodiments comprising a plurality of components are contemplated by the present invention.

Embodiments of the present invention may be constructed of a variety of materials. For example, one embodiment of the present invention may comprise a plastic material. In another embodiment, a fragrance dispenser may comprise metal. In a further embodiment, a fragrance dispenser may comprise both a plastic material and metal.

In one embodiment, a portion of the front face is angled with respect to the back face. For example, in one embodiment as shown in FIG. 1, front face 103 is angled with respect to back face 102 at an angle of approximately 10 to 20 degrees. In other embodiments, front face 103 may be angled with respect to back face 102 at an angle greater than approximately 20 degrees or at an angle less than approximately 10 degrees. In one embodiment, front face 103 may be angled at approximately 0 degrees with respect to back face 102, such that the front face 103 and the back face 102 are substantially parallel. In a further embodiment, the angle may be less than 0 degrees. An angle between the front face 103 and the back face 102 may advantageously expose one or more openings to airflow. For example, as shown in FIG. 2, an angle between the front face 103 and the back face 102 may allow the front face 103 to extend away from a face of a mounting bracket, thus providing a space between the mounting bracket and one or more openings on the front face 103 of the fragrance dispenser. In one embodiment, front face 103 may comprise a substantially frusto-conical shape. Such a configuration may allow increased airflow through openings that may otherwise have been blocked by the front face 103 engaging with the mounting bracket.

In the embodiment shown in FIG. 1, opening 105 in the embodiment shown in FIG. 1 has a substantially circular shape and is located substantially in the center of the front face 103. Other embodiments may comprise an opening having a different shape and/or location. For example in one embodiment, opening 105 may comprise a wedge shape corresponding to a wedge-shaped cut-out in a mounting bracket. In another embodiment, opening 105 may comprise a semi-circular shape having a flat edge. Such an opening may be advantageous for use in a mounting bracket that has a front face with no cut-out or a rectangular-shaped cut out that substantially covers a portion of the front face of the container. Other embodiments of the present invention may comprise an opening 105 having a shape based at least in part on the shape of a mounting bracket or the shape of the container.

In the embodiment shown in FIG. 1, circular opening 105 has a wall 104 enclosing the opening 105. Wall 104 extends from the front face 103 of container 100 and, in some embodiments, may partially or completely encircle opening 105, such as may be seen in the embodiment shown in FIG. 2. Wall 104 may be configured to engage with a cut-out portion of a mounting bracket. In one embodiment, wall 104 may not be adjacent to opening 105, and may be configured to engage with a portion of a mounting bracket. For example, wall 104 may be configured to engage with a mounting bracket, where wall 104 may have an arc shape, wherein the ends of the arc may be adjacent to the outer perimeter of the front face, while the opening may be formed in a different portion of the front face. In the embodiment shown in FIG. 1, wall 104 has an angle of approximately 20 degrees relative to an axis running substantially perpendicular to the back face 102. In other embodiments, wall 104 may have a different angle relative to an axis running substantially perpendicular to the back face 102. For example, in one embodiment, wall 104 may have an angle of greater than approximately 20 degrees relative to an axis running substantially perpendicular to the back face 102. In one embodiment wall 104 may have an angle of less than approximately 20 degrees relative to an axis running substantially perpendicular to the back face 102.

In the embodiment shown in FIG. 1, container 100 comprises a substantially disc shape. Other embodiments of the present invention may comprise other shapes. For example, one embodiment of the present invention may comprise a rectangular shape. Other shapes are contemplated as well, and embodiments having such other shapes may be constructed or used according to the mounting locations, mounting brackets, or other factors.

Container 100 also comprises a plurality of substantially wedge-shaped openings 106 in the front face 103. While a plurality of substantially wedge-shaped openings 106 is shown, an embodiment of the present invention may comprise only one wedge-shaped opening. Another embodiment of the present invention may comprise one or more openings having another shape. For example, one embodiment may comprise one or more ring-shaped openings wherein the one or more ring-shaped openings are concentric with the outer perimeter of the container. Openings having other shapes or a plurality of different shapes may be advantageously employed in still other embodiments. For example, one embodiment may comprise at least one wedge-shaped opening and at least one ring-shaped opening. One embodiment of the present invention may comprise one or more openings along a side face of a fragrance dispenser. For example, container 100 may comprise one or more openings defined by the side face of the container 100.

The number and location of openings in various embodiments of the present invention may be selected based upon the application, location, or other factors. For example, a fragrance dispenser according to one embodiment of the present invention may comprise only one small opening in the front face of the container. Such an embodiment may be well-suited for an environment having a significant airflow, such as a duct or vent, wherein an embodiment having a large number of openings may, when placed in the same location, result in too much fragrance being released too quickly. In contrast, one embodiment of the present invention may comprise a significant number of openings, which may be advantageous in a location having relatively low air flow, such as in the lavatory of an airplane.

FIG. 2 shows a mounting bracket suitable for use with a fragrance dispenser according to one embodiment of the present invention. The mounting bracket 200 shown in FIG. 2 may be a mounting bracket typically used in the airline industry for receiving a fragrance dispenser. As described above, the mounting bracket 200 shown in FIG. 2 comprises a rear face 210, a front face 220 substantially parallel to the rear face 210, a floor 230, and a cut-out 240. Such a mounting bracket 200 may be found in an airplane lavatory and may be mounted to a wall or door.

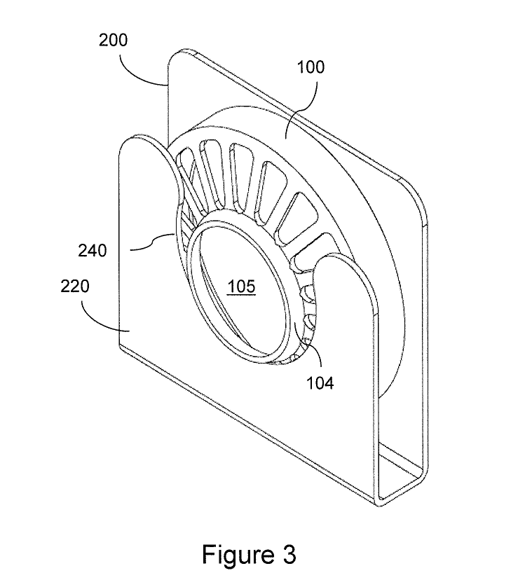

FIG. 3 shows a fragrance dispenser 100 according to one embodiment of the present invention engaged with a mounting bracket 200. In the embodiment shown in FIG. 3, the fragrance dispenser is engaged with the mounting bracket such that a wall 104 surrounding a center opening 105 engages with the cut-out 240 in the front face 220 of the mounting bracket 200. The wall 104 may substantially prevent the fragrance dispenser from rolling out one side of the mounting bracket. For example, if the mounting bracket 200 is mounted inside of an airplane lavatory, the mounting bracket 200 may be tilted or otherwise have its orientation changed during a flight. The fragrance dispenser 100 may remain in place during maneuvers such as take-off, landing, and during turns because the wall 104 may interact with the cut-out 240 to resist movement of the fragrance dispenser 100.

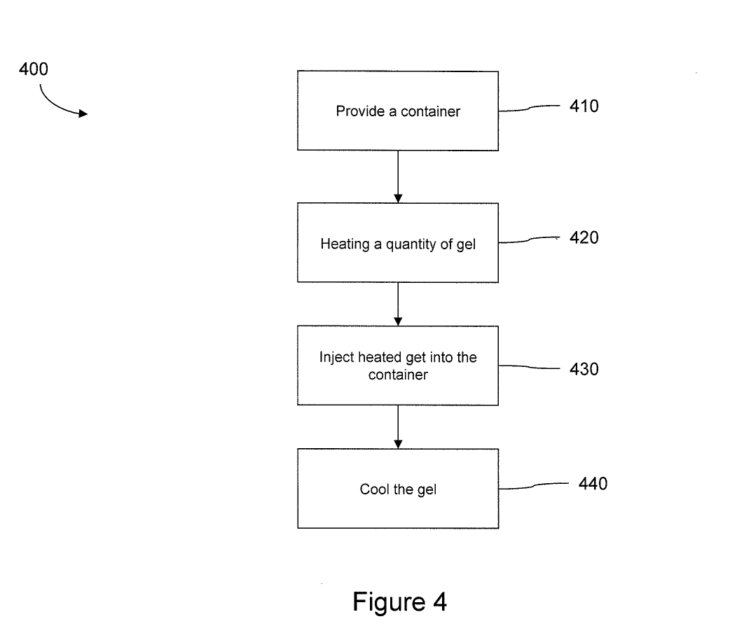

FIG. 4 shows a flow chart illustrating a method for manufacturing a fragrance dispenser according to one embodiment of the present invention. In the embodiment shown in FIG. 4 begins in block 410 with providing a container comprising an inner surface and an outer surface, the inner surface of the container defining a cavity. The inner and outer surfaces define at least one opening that may extend through the outer and inner surfaces, where the opening may be configured to expose the cavity to air. The container may be configured to engage with a mounting bracket.

In block 420, a quantity of gel having a fragrance is be heated such that the gel may be injected into the container. For example, in one embodiment of the present invention, the gel may comprise a gel that is substantially solid at room temperature, but may become liquid when heated above room temperature. In block 430, a quantity of heated gel may be inserted into the container. For example, in one embodiment of the present invention, the quantity of heated gel may be injected into the container. In one embodiment of the present invention, the gel may be poured into the container.

In block 440, the gel is allowed to cool. In one embodiment of the present invention, the cooled gel may solidify and remain in place within the container.

### General

The foregoing description of the embodiments, including preferred embodiments, of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the invention.

That which is claimed is:

1. A device for delivering fragrance, comprising
a container having an inner surface and an outer surface, the inner surface defining a cavity;
a quantity of gel disposed within the cavity, the quantity of gel having a fragrance and configured to evaporate when exposed to air, wherein the container is configured to engage with a mounting bracket; and
at least one opening defined by and extending through the outer and inner surfaces, the opening configured to expose the quantity of gel to air and to provide a visual indication of the quantity of gel.

2. The device of claim 1, further comprising:
a front face defining the at least one opening;
a back face coupled to the front face, wherein the front face and the back face define a cavity.

3. The device of claim 2, wherein the front and back faces each comprise a substantially circular shape, and wherein the front face has a first diameter and the back face has a second diameter.

4. The device of claim 3, wherein the at least one opening comprises a substantially circular opening substantially centered on the front face, the substantially circular opening having a diameter smaller than the diameter of the front face.

5. The device of claim 4, wherein the at least one opening further comprises at least one substantially wedge shape opening located between the substantially circular opening and the edge of the front face.

6. The device of claim 3, wherein the first diameter is substantially the same as the second diameter.

7. The device of claim 3, wherein the first diameter is different from the second diameter.

8. The device of claim 3, wherein the front face has a substantially frusto-conical shape.

9. The device of claim 3, wherein the back face comprises a foot configured to indicate depletion of the gel.

10. The device of claim 1, wherein the container comprises a plastic material.

11. The device of claim 1, wherein the gel comprises a polymer-based gel.

12. A method of manufacturing a device for delivering fragrance, comprising:
providing a container comprising:
an inner surface and an outer surface, the inner surface defining a cavity, and
at least one opening defined by and extending through the outer and inner surfaces, the opening configured to expose the cavity to air, the container configured to engage with a mounting bracket;
heating a quantity of gel having a fragrance;
injecting the quantity of gel into the cavity; and
cooling the quantity gel.

13. The method of claim 12, wherein providing the container further comprises coupling a front face, defining the at least one opening, to a back face to define a cavity.

14. The method of claim 13, wherein the front and back faces each comprise a substantially circular shape, and wherein the front face has a first diameter and the back face has a second diameter.

15. The method of claim 14, wherein the at least one opening comprises a substantially circular opening substantially centered on the front face, the substantially circular opening having a diameter smaller than the diameter of the front face.

16. The method of claim 15, wherein the at least one opening further comprises at least one opening having a substantially wedge shape and located between the substantially circular opening and the edge of the front face.

17. The method of claim 14, wherein the first diameter is substantially the same as the second diameter.

18. The method of claim 14, wherein the first diameter is different from the second diameter.

19. The method of claim 14, wherein the front face has a substantially frusto-conical shape.

20. The method of claim 14, wherein the back face comprises a foot configured to indicate depletion of the gel.

21. The method of claim 12, wherein the container comprises a plastic material.

22. The method of claim 12, wherein the gel comprises a polymer-based gel.
